(19) United States
(12) Patent Application Publication
(10) Pub. No.: US 2024/0298992 A1
(43) Pub. Date: Sep. 12, 2024

(54) COMPUTER-IMPLEMENTED METHOD FOR PREPARING A COMPUTED TOMOGRAPHY SCAN, COMPUTER PROGRAM, COMPUTER-READABLE STORAGE MEDIUM, AND COMPUTED TOMOGRAPHY SYSTEM

(71) Applicant: Siemens Healthineers AG, Forchheim (DE)

(72) Inventors: Matthias BAER-BECK, Spardorf (DE); Andreas PROKEIN, Bubenreuth (DE); Thomas ALLMENDINGER, Forchheim (DE); Martin PETERSILKA, Adelsdorf (DE)

(73) Assignee: Siemens Healthineers AG, Forchheim (DE)

(21) Appl. No.: 18/599,676

(22) Filed: Mar. 8, 2024

(30) Foreign Application Priority Data
Mar. 10, 2023 (EP) 23161129.4

(51) Int. Cl. A61B 6/00; A61B 6/03; A61B 6/40; G06T 7/62

(52) U.S. Cl. CPC A61B 6/5258; A61B 6/032; A61B 6/4035; A61B 6/544; G06T 7/62; G06T 2207/10081; G06T 2207/30004

(57) ABSTRACT

A computer-implemented method for preparing a computed tomography scan of a subject, the method comprising: receiving scan-related information including subject-related information; automatically adapting at least two imaging parameters based on seeking to optimize image quality via an optimization function, wherein the optimization function includes the scan-related information as at least one constant and the imaging parameters as variables to be optimized, and wherein optimizing image quality includes reducing scan artifacts of the computed tomography scan; and providing the adapted imaging parameters.

### CROSS-REFERENCE TO RELATED APPLICATION (S)

The present application claims priority under 35 U.S.C. § 119 to European Patent Application No. 23161129.4, filed Mar. 10, 2023, the entire contents of which are incorporated herein by reference.

### FIELD

One or more example embodiments of the present invention relate to a computer-implemented method for preparing a computed tomography scan and a corresponding non-transitory computer-readable medium, and/or computed tomography system.

### BACKGROUND

Independent of the grammatical term usage, individuals with male, female or other gender identities are included within the term.

With the technical development in medical imaging also come higher demands are made regarding image quality. On the one hand, image quality, in particular in computed tomography (CT) imaging, can be influenced by the technical side, such as by the quality of the hardware. On the other hand, image quality can also be influenced by the choice of parameters that are used during the imaging process. Image quality may, for example, be characterized by a contrast-to-noise ratio. For example, in CT imaging, some scan parameters may be selected such that a contrast-to-noise ratio is optimized. A contrast-to-noise ratio usually is defined as a ratio of a difference between two signal intensities in a region of interest, in particular as numerator, and of a value describing image noise, in particular as denominator. The value describing the image noise may for example be a standard deviation of image noise. Image noise may be defined to be a random variation of the image signal, e.g., a random variation of a grey value, or of a colour value. Image noise may, for example, be created by electronic noise in the used hardware parts or in the power supply lines of the surrounding building or by mechanical vibrations.

In order to provide a good image quality for each respective demand, in the state of the art, a user may typically choose a scan protocol or a basic scan mode depending on what is to be examined and what kind of hardware is available. Hence, a user may choose a scan protocol that seems most suitable to produce good imaging data of an object or subject to be examined. The chosen scan protocol may then be further adapted to optimize the contrast-to-noise ratio.

However, in particular with advancing progress of technical possibilities, the demands of image quality are also increasing. Thus, it would be desirable to increase image quality even further. An option may be to provide a wider array of scan protocols and scan modes to the user, such that, having more choice, the user may find a more optimized solution for each individual case. However, having more options may also increase the complexity of imaging, and may thus lead to more problems and difficulties regarding maintenance and handling of a corresponding imaging system. Accordingly, the demands on the user's expertise may increase with the complexity of the imaging system, making it more difficult to run such an imaging system.

### SUMMARY

It is therefore an object of one or more example embodiments of the present invention to provide a mechanism and/or means to improve image quality.

At least this object is met or exceeded by at least a method, a non-transitory computer-readable medium, and/or a computed tomography system according to one or more embodiments of the present invention.

According to a first aspect of embodiments of the present invention, a computer-implemented method for preparing a computed tomography (CT) scan of a subject is provided. The method comprises the following steps:
(a) receiving scan-related information comprising subject-related information;
(b) automatically adapting at least two imaging parameters in order to optimize image quality via an optimization function, wherein the optimization function comprises the scan-related information as at least one constant and the imaging parameters as variables to be optimized, wherein optimizing image quality comprises reducing scan artifacts of the computed tomography scan;
(c) providing the adapted imaging parameters.

The subject may in particular be a human being or animal, in particular a patient. Scan-related information may preferably be defined as information that is relevant for the CT scan, in particular for the image quality. The scan-related information comprises subject-related information, i.e., information about the subject that is related to the scan. Hence, subject-related information may preferably be defined to be information about the subject that is relevant for the CT scan, in particular for the image quality. Subject-related information may be patient-related information, e.g., when the subject is a patient. The scan-related information may, for example, comprise a contrast-related information, such as body tissue characteristics and/or contrast medium concentration. Subject-related information may, for example, comprise geometric information of the subject and/or an age or weight of the subject. Geometric information may, for example, concern a size or diameter of the subject.

Imaging parameters may preferably comprise scan parameters and reconstruction parameters. In some cases, it may be feasible to only take into account either scan parameters or reconstruction parameters. However, in most cases, taking into account both will be advantageous, e.g., to achieve a more well-rounded optimization of image quality. Scan parameters are in particular parameters that are applied during a CT scan, such as tube voltage, filter setting, tube current, and detector collimation, to acquire raw data. For example, tube voltage may determine the photon energy of the X-ray beam and thus influence the contrast but also the noise. Usually, a CT system provides the possibility to set different tube voltages for a CT scan. Increasing tube current may reduce noise but may also increase a radiation dose. Reconstruction parameters, such as setting the keV level of monoenergetic image reconstructions in spectral scan modes, are in particular parameters that are applied to reconstruct image data from the acquired raw data. Setting keV levels may, for example, be used to enhance the contrast between different materials. In particular, during reconstruction, several slices of the subject may be calculated which, put together, may result in three-dimensional image data of the subject.

The optimization function comprises the scan-related information and the imaging parameters. Hence optimizing the optimization function may be done with regard to the scan-related information and the imaging parameters. Since the scan-related information is provided as a constant and the imaging parameters are a variable, optimizing the optimization function may in particular comprise adjusting the imaging parameters, preferably such that the optimization function is minimized or that the optimization function is maximized. The optimization function may for example be a cost function. For example, assuming scan and reconstruction parameters {p_{scan}; p_{recon}} as imaging parameters, the adapted imaging parameters may comprise optimized scan and reconstruction parameters {p_{scan}^{opt}; p_{recon}^{opt}}. Preferably, p_{scan} and p_{recon} may both be vectors, each containing a collection of one or several scan parameters or one or several reconstruction parameters, respectively. Hence, exemplarily, the adapted imaging parameters may be determined by minimizing a cost function in the form of

{p_{scan}^{opt};p_{recon}^{opt}}=argmin {Cost(p_{scan},p_{recon},c)},

where c is a constant describing the scan-related information regarding the subject. Therein, “Cost” is a cost function that is to be optimized by minimizing it via adjusting the imaging parameters {p_{scan}; p_{recon}} while c is kept constant. The imaging parameters may for example be a keV level for reconstruction [keV], a wedge filter setting [WF], a tube voltage [U] and a tube current [mAs], such that the function for seeking to optimize the cost function may take the form

{keV^{opt},WF^{opt},U^{opt},mAs^{opt}}=argmin {Cost(keV,WF,U,mAs,c)}.

The optimization function may be designed to only optimize scan quality via reducing scan artifacts. For example, the cost function Cost may be a cost function S_{Artifact} designed to reduce artifacts. For example, the cost function S_{Artifact} may be generated empirically by running phantom scans and assessing the image quality of thereby generated images by using physical models of the imaging and reconstruction process or by a combination of both. Alternatively, the optimization function may be designed to optimize multiple aspects of scan quality, such as reducing scan artifacts and increasing a contrast-to-noise ratio. It may be an option to decouple the optimization of different aspects of image quality, in particular of reducing scan artifacts and of another aspect of image quality, such as contrast-to-noise ratio. Hence, there may be two steps of optimization, wherein first, a first aspect of image quality is optimized, thereby adapting at least one first imaging parameter, and then, a second aspect of image quality is optimized, thereby adapting at least one second imaging parameter, while the at least one first imaging parameter is kept fixed. In particular, for example, the first aspect may be a contrast-to-noise ratio, and the second aspect may be reducing scan artifacts, such as scatter artifacts. The at least one first imaging parameter may for example be one or several of the following: a tube current profile, a tube voltage, a keV level. The at least one second imaging parameter may for example be a wedge filter setting.

The optimization function is preferably designed such that its optimization leads to an improved image quality. Image quality may be a measure for reliability of the acquired image data and/or information derivable from the acquired image data. Image quality may be characterized by different aspects. Aspects of image quality may, for example, comprise image contrast, image noise and spatial resolution. Usually, a higher image contrast as well as lower noise level is desirable. Thus, depending on the clinical task as well as the boundary conditions thus as an applied contrast agent, a measure of image quality that may be used is the contrast-to-noise ratio. According to embodiments of the present invention, an aspect of image quality are in particular scan artifacts. Scan artifacts may, for example, be caused by scattering, such as forward scattering or cross scattering. Cross scattering may in particular occur in Dual Source CT. Further causes of scan artifacts may be photon starvation, beam hardening, e.g., due to metals, cone beam with respect to detector collimation, movement of the subject, and/or spectral separation. The inventors have found that with increasing technical standards of CT imaging, scan artifacts can have a larger impact on image quality, in particular in terms of reliability, then previously thought. Usually, according to the state of the art, there is not provided an automatic adaption of imaging parameters based on (potential) scan artifacts. Here, it has been found that, especially in non-standard circumstances such as particularly large subjects, scan artifacts can play a significantly important role for image quality that should advantageously not be neglected. Taking into account scan artifacts may lead to a more complex calculation of imaging parameters. Furthermore, an optimization of the imaging parameters with regard to scan artifacts may, for example, potentially lead to a less optimized contrast-to-noise ratio because the imaging parameters are determined based on additional restraints, namely to reduce scan artifacts. Nevertheless, it has been found that, depending on the subject to be examined, the optimization with regard to scan artifacts may still lead to a better overall image quality, in particular with respect to reliability and/or information contained in the image data. Thus, it is advantageous to optimize scan quality (at least partly) based on seeking to reduce scan artifacts in order to take into account effects of image artifacts.

The method may, for example, be carried out by a processing unit such as a CPU, a GPU, a computer or part of a computer. The computer may be a PC, a server, and/or a control unit of a computed tomography (CT) system. The computer may also be a mobile device, such as a laptop, tablet computer or mobile phone. The control unit may in particular comprise a dedicated workflow engine, e.g., comprising a computer program configured to carry out the corresponding method steps. The adapted imaging parameters may in particular be provided to the CT system before the start of a CT scan.

According to an embodiment, the optimization function comprises a linear combination of a plurality of sub-cost functions, each sub-cost function representing another aspect of image quality. In particular, each sub-cost function may be designed such that optimizing it leads to reducing the corresponding aspect of image quality. In particular, the optimization function may be a cost function comprising the linear combination of a plurality of sub-cost functions. Accordingly, seeking to optimize the optimization function may lead to reducing the overall image quality taking into account the different aspects of image quality. An aspect of image quality may be defined such that it is a criterion that influences image quality. One aspect of image quality is in particular an occurrence of image artifacts. Further aspects of image quality may, for example comprise one or several of the following: an image contrast, image noise, in particular a contrast-to-noise ratio, and a spatial resolution. The sub-cost functions may each have a pre-factor. The pre-factors may be designed to normalize the different sub-cost functions and/or to weight the different sub-cost functions with respect to each other. For example, the sub-cost functions may be weighted with respect to their impact on the overall image quality. Considering several sub-cost functions costᵢ, the above-mentioned cost function Cost may thus, for example, take the form

$$Cost ( {p}_{scan} , {p}_{recon} , c ) = {\sum }_{i} {λ}_{i} \cdot {cost}_{i} ( {p}_{scan} , {p}_{recon} , c ) ,$$

where λᵢ are the pre-factors of the different sub-cost functions costᵢ. Therein, each sub-cost functions costᵢ describes an aspect of image quality that is to be optimized. Preferably, one of the sub-cost functions cost; may be a cost function designed to reduce scan artifacts S_{Artifact}. For example, another sub-cost function may be a cost function C_{CTNR} that is designed to increase the contrast-to-noise ratio.

According to an embodiment, the optimization function comprises at least one penalty term, preferably a plurality of penalty terms. According to an embodiment, at least one of the plurality of sub-cost functions is or comprises a penalty term, the penalty term in particular being weighted by a weighting parameter. The weighting parameter may correspond to the pre-factor mentioned above. It may be provided that several of the sub-cost functions are or comprise a penalty term. The at least one penalty term or, respectively, each penalty term may comprise at least one of the imaging parameters as a penalty parameter. The weighting parameter may be chosen such that it corresponds to a measure of the impact of the corresponding at least one imaging parameter on the overall image quality. Advantageously, the penalty term or preferably a plurality of penalty terms may be an efficient way of considering different aspects of image quality. According to an embodiment, at least one penalty term relates at least one of the scan-related information to at least one of the imaging parameters. For example, a patient size may be related to a wedge filter setting or to a CT scan mode to be chosen. Hence, advantageously, the corresponding imaging parameter may be optimized with regard to the at least one scan-related information.

According to an embodiment, the optimization function comprises a dose response function, the dose response function depending on the at least two imaging parameters. The dose response function may correspond to a radiation dose that is applied to the subject during a CT scan. The dose response function may be designed such, that a Computed Tomography Dose Index (CTDI) is minimized during optimization. The CTDI is a standard measure of dosimetry and suitable as basis for the calculation of the radiation exposure of the subject during the CT scan. Due to detrimental effects on health of the subject caused by radiation, it is usually a goal to minimize the radiation dose the subject is exposed to as much as possible. On the other hand, an increased radiation dose may be necessary to improve image quality, e.g., by increasing the tube current. Accordingly, providing the dose response function as part of the optimization function may advantageously allow to take this aspect into account when adapting the imaging parameters. The dose response function may be of the form

D(p_{scan},p_{recon},c).

In particular the dose response function may be part of the linear combination of sub-cost functions, representing an additional cost function. The dose response function may, optionally, not be designed to improve image quality but to reduce radiation dose. However, alternatively, the dose response function may preferably be designed to take into account both, a reduction of a radiation dose and an optimization of contrast-to-noise ratio. For example, the cost function may thus be in the form of

$$Cost ( {p}_{scan} , {p}_{recon} , c ) = α D ( {p}_{scan} , {p}_{recon} , c ) + {\sum }_{i} {λ}_{i} \cdot {cost}_{i} ( {p}_{scan} , {p}_{recon} , c ) ,$$

where α is the pre-factor of the dose response function and costᵢ are the remaining sub-cost functions with pre-factors λᵢ. Preferably the dose response function depends on all imaging parameters that are considered in the entire cost function, i.e., in all sub-cost functions.

According to an embodiment, the scan-related information comprises a size of the subject. In particular, if the subject is a patient, the size of the subject may be a patient size. The patient size may preferably be determined automatically. For example, the subject size may be derived from a topogram that is taken at the beginning of the CT examination in which the CT scan whose imaging parameters are adapted is to be acquired. A topogram is typically a 2-dimensional X-ray image acquired using a CT scanner. The topogram is routinely used in clinical CT scanning to define the scan range of the subsequent CT scan. Alternatively, the subject size may be determined from a camera image, in particular a camera image taken at the beginning of the CT examination in which the CT scan, whose imaging parameters are adapted, is to be acquired. E.g., the CT system may comprise a camera and be configured to automatically take a camera image of the subject and determine the size of the subject from the camera image. The camera may be a 2D camera or a 3D camera. It may be installed above the patient bed, for example fixed at the ceiling. Alternatively, the patient size may be input by a user or retrieved from a database. According to an embodiment, the scan-related information comprises a diameter of the subject at a region of interest that is to be scanned. Hence, the subject size may be defined by the diameter of the subject. It has been found that large patients may lead to particularly strong artifacts at least in some kind of CT imaging examinations, e.g., during cardiac CT imaging with photon counting detectors. This can be explained by a combination of effects. First, primary intensities of the CT imaging signal may be particularly low for large subjects due to damping of the signal in the subject, e.g., in a patient's body. Further, also a scatter intensity typically increases with subject size, since a larger subject diameter leads to a longer way of the x-ray beam through the subject and thus to more opportunities for scattering. Both these factors are typically exponentially dependent on the subject's diameter. Hence, particularly large subjects can lead to strong artifacts, whereas basic models assuming an averaged size subject may not show any significant impact of scan artifacts. Hence considering both the subject size and its impact on scan artifacts may advantageously lead to an improved image quality, to an even greater effect than more basic models might indicate.

According to an embodiment, the imaging parameters comprise a wedge filter setting. The wedge filter setting may, for example, concern the geometric properties of a wedge filter. The wedge filter setting may concern a width of the wedge filter. Usually, according to the state of the art, a wedge filer, e.g., a cardio wedge, is designed that a resulting dose is chosen with regard to the majority of subjects. However, in particular for very large subjects or subjects with unusual anatomy, it has been found that such a standard wedge may be non-optimal for image quality in some cases. For example, while large subjects or unusual anatomy, such as implants, may lead to reduced imaging signal intensities, these already reduced intensities are further reduced by the wedge filter. Additionally, also the scatter intensity increases with the subject's size. The decreased signal intensity, due to subject size and wedge filter combined, may now be so low that a decrease in scatter intensity due to the wedge filter may no longer be enough to result in a net positive effect of the wedge filter with regard to the image quality. For example, a large subject size may lead to a significant increase in scattering being directed essentially parallel to the x-ray beam, e.g., forward scattering that is not filtered as efficiently as cross scattering by the wedge filter. Hence, in this case it may be beneficial for the image quality to use another wedge filter, e.g., with a wider width, to increase the imaging signal intensity.

According to an embodiment, adapting imaging parameters comprises, depending on the scan-related information, in particular depending on the subject size, determining a wedge filter to be used out of a plurality of at least two available wedge filters. The plurality of wedge filters may preferably differ from each other. In particular the geometric properties of the applied wedge filter may be set by choosing from the plurality of available wedge filters, wherein the available wedge filters differ from each other with respect to their geometric properties. Additionally and/or alternatively the wedge filters may differ with respect to their material properties. For example, a typical wedge filter used for cardio imaging, i.e., a cardio wedge, may have a shape that is designed such that a dose distribution is focused to an inner part of the scan field of view of the scanner, namely the heart region. This can be achieved by providing an increasing thickness of the wedge with increasing distance from the isocenter. This may allow, that regions are located in the periphery of the x-ray fan, e.g., the lungs, receive less radiation exposure. While such a standard geometry may be suitable for the majority of subjects, the geometry may be less optimal for unusual subjects, e.g., particularly large subjects. Namely, the increasing thickness of the wedge that reduces the dose towards an outer region may also increase the ratio of scatter to signal intensity of photons reaching the CT detector. This may lead to a higher scatter artifact level in the reconstructed image. Accordingly, it may be advantageous to replace the standard cardio wedge with another wedge filter more appropriate for this particular subject. For example, a wedge filter that has a muss less pronouncedly shaped profile may be more beneficial for large subjects. This may lead to the signal intensities in the periphery being increase and therefore the scatter to signal intensity ratio and, consequently, the artifacts in this region may be reduced as well.

According to an embodiment, adapting imaging parameters comprises choosing a computed tomography (CT) scan mode out of a plurality of available computed tomography scan modes. A CT scan mode to be chosen from may for example be a dual source mode versus a single source mode or a multi energy mode versus a single energy mode. Further CT scan modes may be spectral scan modes, such as TwinBeam, TwinSpiral, or Photon Counting modes. For example, the CT scan mode may be optimized to achieve a desired spectral separation needed for a specific task, such as monoenergetic, VNC, bone marrow. Preferably the optimization may be based on a subject size, in particular a subject diameter. For example, while dual source CT has some particular, well-known benefits versus single source CT, the overall scatter intensity tends to be higher due to cross scattering from the first source to the second detector and from the second source to the first detector. This may impact a negative effect of a standard cardio wedge filter as described above even more.

According to an embodiment, the imaging parameters are scan parameters comprising at least one of the following: a kV filter setting, a spectral filter setting, a collimation setting, a spiral pitch factor and a rotation time setting. Higher kV filter settings may enable to reduce beam hardening or photon starvation artifacts. Higher kV levels may be particularly beneficial for large subject diameters. A removal of spectral filters may be beneficial for large subject diameters. The detector collimation may in particular influence a spatial resolution. A smaller collimation size usually leads to a higher spatial resolution and may reduce artifacts, such as scatter artifacts. However, a smaller detector collimation may also lead to an increased noise. Hence, there are conflicting effects of the collimation size. It has been found that these effects may depend heavily on the subject size. The spiral pitch factor may determine the scan speed and a speed of a patient table moving through the CT scanner. The spiral pitch factor and/or the rotation time setting may be adapted depending on the subject size. This may be beneficial in order to reduce scan artifacts.

According to a further aspect of embodiments of the present invention, the imaging parameters comprise at least one reconstruction parameter, the at least one reconstruction parameter being a keV level of reconstruction. The keV level of reconstruction may have an impact on both scan artifacts and a contrast-to-noise ratio. For example, photon counting CT detectors, may enable to apply dual energy image reconstruction methods, allowing the generation of monochromatic images, in particular also in the field of cardiac CT scanning. The optimal keV level at which monochromatic images of angiographies are displayed is typically in the low energy regime, e.g., between 50 keV and 60 keV. Relatively to the primary x-rays, namely the x-rays contributing to the signal intensity, scattered x-rays typically have a lower energy level since they have lost parts of their energy during the scattering. If images are reconstructed at lower levels of keV, this may increase the scatter to primary x-ray ratio and thereby the scatter artifact level in the reconstructed images. Since the keV level of the reconstruction thus may impact the final image quality with respect to scatter artifacts it may be desirable to include this reconstruction parameter in the optimization calculation.

The embodiments described herein may be combined with each other unless indicated otherwise. For example, combining several of the above embodiments, a cost function or sub-cost function designed to reduce scan artifacts S_{Artifact}, may regard a patient size, in particular patient diameter d_{Patient} and a wedge filter setting WF. A further sub-cost function C_{CTNR} may be designed to increase the contrast-to-noise ratio. An overall optimization function for a combined optimization of a wedge filter setting, the keV level of reconstruction, and a contrast-to-noise ratio on a photon counting CT system may take the form

$$Cost ( keV , WF , U , mAs c ) = {λ}_{1} \cdot {S}_{Artifact} ( keV , {d}_{Patient} , WF ) + {λ}_{2} \cdot {C}_{CTNR} ( MC , U , {d}_{Patient} , keV , mAs )$$

with the pre-factors λ₁ and λ₂ that are used to weight the impact of the two cost functions. Via the pre-factors the tradeoff between the optimization with regard to artifacts and the optimization with regard to contrast-to-noise ratio may be influenced and controlled. MC is a material contrast, U is a tube voltage, and mAs is a tube current. Therein, [keV] may be a reconstruction parameter, [WF, U, mAs] may be scan parameters and [MC, d_{Patient}] may be scan-related information that are kept constant when calculating the optimization. Advantageously, in this particular example, applying the linear combination allows to optimize the keV level because the keV level has an impact on both the artifacts and the contrast-to-noise ratio. A low keV level may lead to higher scatter artifacts and, at the same time to a better iodine-water contrast-to-noise ratio. Similarly, applying a linear combination may be beneficial in other cases, where an imaging parameter has an effect on different aspects of image quality. In an alternative example, the optimization of the image quality may comprise reducing the scan artifacts separately. This may for example be an option, when the keV level of reconstruction is determined with respect to the contrast-to-noise ratio and is thus not used as a variable in order to reduce scan artifacts. In this case, the cost function may, for example, be of the form

Cost(WF,c)=S_{Artifact}(d_{Patient},WF)

According to a further aspect of embodiments of the present invention, a non-transitory computer-readable medium storing a computer program including instructions which, when the program is executed by a computer, cause the computer to carry out the method as described herein. All features and advantages of the method may be adapted to the computer program stored on non-transitory computer-readable medium, and vice versa.

According to a further aspect of embodiments of the present invention, a computer-readable storage medium, in particular non-transient storage medium, is provided that comprises instructions which, when executed by a computer, cause the computer to carry out the method as described herein. All features and advantages of the method and of the computer program may be adapted to the computer-readable storage medium and vice versa.

According to a further aspect of embodiments of the present invention, a computed tomography (CT) system comprising a processing unit that is configured to carry out the method as described herein is provided. All features and advantages of the method, of the computer program and of the computer-readable storage medium may be adapted to the computed tomography system and vice versa.

### BRIEF DESCRIPTION OF THE DRAWINGS

The accompanying drawings illustrate various exemplary embodiments and methods of various aspects of the present invention.

FIG. 1 shows a flow diagram of a method according to an embodiment of the present invention;

FIG. 2 shows examples of relations between scan-related information and imaging parameters of four penalty terms;

FIG. 3 depicts the problem of cross scattering in dual source mode;

FIG. 4 shows a computed tomography system 1 according to an embodiment of the present invention.

Similar elements are designated with the same reference signs in the drawings.

### DETAILED DESCRIPTION

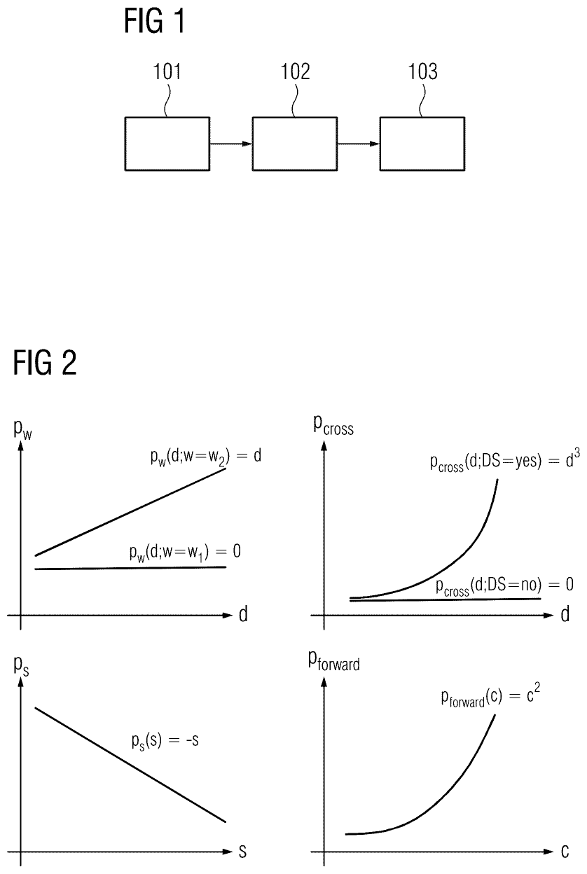

FIG. 1 shows a flow diagram of a method according to an embodiment of the present invention. The method is designed for preparing CT scan of a subject 5. Therein, in a first step 101, scan-related information comprising subject-related information is received by a processing unit 2 of a CT system 1. The subject-related information may in particular comprise a size of the subject 5, e.g., defined by a diameter of the subject 5. In a further step 102, at least two imaging parameters are adapted based on seeking to optimize image quality via an optimization function. The imaging parameters may for example be a wedge filter setting, a kV filter setting, a spectral filter setting, a collimation setting, a spiral pitch factor, a rotation time setting, or a a keV level of reconstruction. The optimization function comprises the scan-related information as at least one constant and the imaging parameters as variables to be optimized. In the optimization step, optimizing the image quality comprise reducing scan artifacts of the computed tomography scan. In a further step 103 the adapted imaging parameters are provided, e.g., to be used during a following CT scan and image reconstruction.

According to an embodiment, the optimization function comprises a linear combination of a plurality of sub-cost functions each sub-cost function representing another aspect of scan image quality. The sub-cost functions are in the form of penalty terms that are weighted by pre-factors that serve as weighting parameters. The optimization function further comprises a dose response function D that depends on the imaging parameters and that is also part of the linear combination in this example. Preferably optimizing may be achieved by minimizing the linear combination, which may for example have the following form:

$$arg min [ α D ( kV ; c ; {w}_{x} ) +$$

$$β {p}_{w} ( d ; {w}_{x} ) + γ {p}_{s} ( s ) + δ {p}_{cross} ( d , DS ) + ε {p}_{forward} ( c ) ]$$

Hence, there is a linear combination of the dose response function D and several penalty terms p. The dose response functions and the penalty terms are weighted by pre-factors (α,β,γ,δ,ε). The penalty terms are depicted as functions in FIG. 2. A first penalty term, p_{w}, describes two different variants of wedge filters having different widths, namely w₁ and w₂. The wedge filters may differ in further characteristics, e.g., their overall geometric shape. The wedge filters are plotted against a subject diameter, d. As can be seen, the course of the graph is different for both wedge filters. This difference, i.e., the respective (linear) dependency with regard to the subject diameter is put into the penalty term. This penalty term or another penalty term (not included in this example) may further comprise information about a contrast-to-noise ratio influenced by the wedge setting. Another penalty term, p_{cross}, describes a dependence of cross scattering on the patient diameter, d, both for dual source (DS=yes) and for single source (DS=no). Accordingly, depending on patient size single source or dual source may be more beneficial for image quality. Another penalty term, pₛ, generally defines a faster acquisition speed, s, as favorable, thus increasing the penalty for lower speed (in this case, the relation is inversely linear). This penalty term may be used to aim for a faster acquisition speed, as far as this is feasible, e.g., to avoid getting to slow acquisition speeds via this method. Another penalty term, p_{forward}, describes the dependency of forward scattering on a collimator width, c, which increases exponentially to the power of 2 in this example. The given penalty terms are to be understood as examples. Further penalty terms may be added to further refine the optimization.

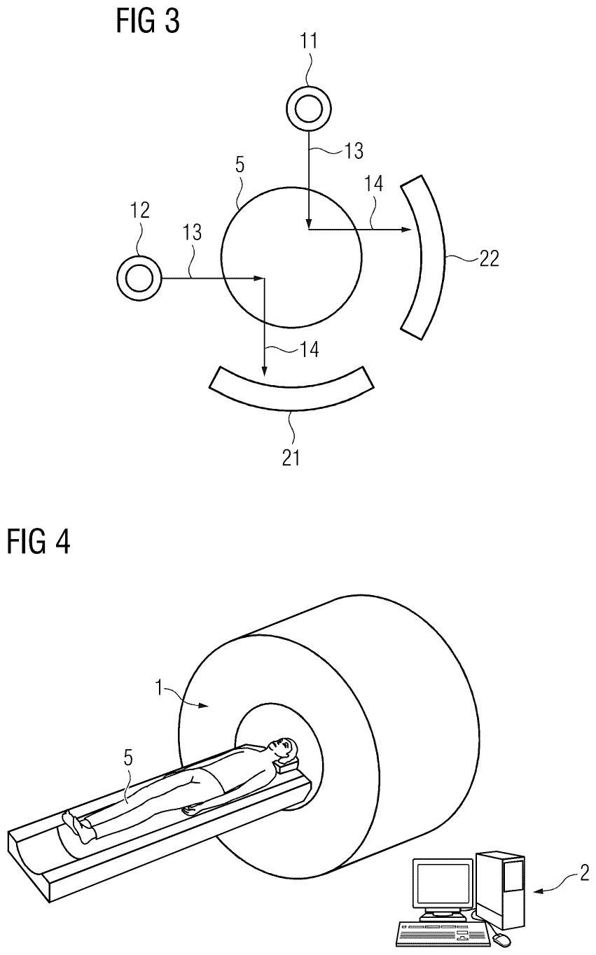

FIG. 3 depicts the problem of cross scattering in dual source mode. In dual source mode, two x-ray sources, a first x-ray source 11 and a second x-ray source 12 are used together with a corresponding first detector 21 and second detector 22. Primary x-rays 13 from the first source 11 are detected by the first detector 21 and primary x-rays 13 from the second source 12 are detected by the second detector 22. However, due to scattering in the object 5, there are also scattered x-rays 14. Due to this cross scattering some scattered x-rays 14 from the first source 11 also reach the second detector 22 and some scattered x-rays 14 from the second source 12 also reach the first detector 21, leading to cross-scatter artifacts. This effect can be significantly stronger for a subject 5 with a large diameter, since the longer way of the primary x-rays through the subject 5 leads to more opportunities for scattering and thus to the occurrence of more cross scattering.

FIG. 4 shows a computed tomography (CT) system 1 according to an embodiment of the present invention. The CT system 1 comprises a processing unit 2 that is configured to carry out the method as described herein. The processing unit 2 in this example is depicted as a personal computer. The processing unit 2 may in particular be a control unit of the CT system 1.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element (s) or feature (s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for preparing a computed tomography scan of a subject, the method comprising:
receiving scan-related information including subject-related information;
automatically adapting at least two imaging parameters via an optimization function in order to optimize image quality, wherein the optimization function includes the scan-related information as at least one constant and the at least two imaging parameters as variables to be optimized, and wherein optimizing of the image quality includes reducing scan artifacts of the computed tomography scan; and
providing the adapted at least two imaging parameters.

2. The computer-implemented method according to claim 1, wherein the optimization function comprises a linear combination of a plurality of sub-cost functions, each of the plurality of sub-cost functions representing another aspect of image quality.

3. The computer-implemented method according to claim 2, wherein at least one of the plurality of sub-cost functions is a penalty term, the penalty term being weighted by a weighting parameter.

4. The computer-implemented method according to claim 3, wherein at least one penalty term relates at least a portion of the scan-related information to at least one of the imaging parameters.

5. The computer-implemented method according to claim 1, wherein the optimization function comprises a dose response function, the dose response function depending on the at least two imaging parameters.

6. The computer-implemented method according to claim 1, wherein the subject is a patient, and the scan-related information comprises a size of the patient.

7. The computer-implemented method according to claim 1, wherein the scan-related information comprises a diameter of the subject at a region of interest that is to be scanned.

8. The computer-implemented method according to claim 6, further comprising:
receiving a topogram or a camera image of the subject, wherein the topogram or the camera image has been acquired at a beginning of a CT examination in which a CT scan whose imaging parameters are adapted is to be acquired; and
deriving a size or diameter of the subject from the topogram or the camera image.

9. The computer-implemented method according to claim 1, wherein the at least two imaging parameters comprise a wedge filter setting.

10. The computer-implemented method according to claim 1, wherein automatically adapting the at least two imaging parameters comprises:
determining, depending on the scan-related information, a wedge filter to be used out of at least two available wedge filters.

11. The computer-implemented method according to claim 1, wherein the automatically adapting the at least two imaging parameters comprises:
choosing a computed tomography scan mode from a plurality of available computed tomography scan modes.

12. The computer-implemented method according to claim 1, wherein
the at least two imaging parameters are scan parameters, and
the scan parameters include at least one of a kV filter setting, a spectral filter setting, a collimation setting, a spiral pitch factor or a rotation time setting.

13. The computer-implemented method according to claim 1, wherein the at least two imaging parameters comprise at least one reconstruction parameter, the at least one reconstruction parameter being a keV level of reconstruction.

14. A non-transitory computer-readable medium storing computer-readable instructions that, when executed by a computer, cause the computer to carry out the computer-implemented method of claim 1.

15. A computed tomography system comprising:
at least one processor configured to carry out the computer-implemented method according to claim 1.

16. The computer-implemented method according to claim 2, wherein at least one of the plurality of sub-cost functions is a penalty term.

17. The computer-implemented method according to claim 1, wherein the scan-related information comprises a size of the subject.

18. The computer-implemented method according to claim 8, wherein the at least two imaging parameters comprise a wedge filter setting.

19. The computer-implemented method according to claim 2, wherein the automatically adapting the at least two imaging parameters comprises:
determining, depending on the scan-related information, a wedge filter to be used out of at least two available wedge filters.

20. The computer-implemented method according to claim 5, wherein the automatically adapting the at least two imaging parameters comprises:
determining, depending on the scan-related information, a wedge filter to be used out of at least two available wedge filters.
